(19) United States
(12) Patent Application Publication
(10) Pub. No.: US 2009/0036464 A1
(43) Pub. Date: Feb. 5, 2009

(54) Treatment Of Posterior Capsule Opacification

(75) Inventors: Barbara Ann Spruce, Perthshire (GB); George Duncan, Norfolk (GB); Peter Reid Duncan, Aberdeen (GB); Alan Prescott, Fife (GB)

Correspondence Address: MYERS BIGEL SIBLEY & SAJOVEC, PO BOX 37428, RALEIGH, NC, 27627, US

(73) Assignee: UNIVERSITY COURT OF THE UNIVERSITY OF DUNDEE, Dundee (GB)

(21) Appl. No.: 11/661,482

(22) PCT Filed: Aug. 26, 2005
(86) PCT No.: PCT/GB2005/003373

§ 371 Date: Nov. 14, 2007

(30) Foreign Application Priority Data
Aug. 27, 2004 (GB) 0419145.8

(51) Int. Cl. C12Q 1/02; A61K 31/496; A61K 31/135; A61P 27/02

(52) U.S. Cl. 514/253.03; 435/29; 514/646

(57) ABSTRACT

This invention relates to a method of testing agents, such as sigma ligands for their potential use in treating posterior capsule opacification (PCO). The present invention also relates to the use of sigma ligands, such as sigma-1 antagonists in the manufacture of a medicament for preventing posterior capsule opacification (PCO), as well as methods of treating PCO using sigma ligands.

### FIELD OF THE INVENTION

The invention relates to a method of testing agents, such as sigma ligands for their potential use in treating posterior capsule opacification (PCO).

The present invention also relates to the use of sigma ligands, such as sigma-1 antagonists in the manufacture of a medicament for preventing posterior capsule opacification (PCO), as well as methods of treating PCO using sigma ligands.

### BACKGROUND TO THE INVENTION

Cataract extraction, followed by artificial lens implantation, is the commonest surgical procedure in the Western world. It is, however, beset by the major problem that in approximately 30-50% of cases the procedures need to be repeated, sometimes multiple times, due to a post-operative complication known as posterior capsule opacification (PCO) which is more common in younger patients (almost 100%). Additionally secondary intervention by Nd-YAG laser can itself result in further complications. PCO arises from the inappropriate growth of lens epithelial cells that normally line the anterior face of the “capsule”—the bag within which the lens lies. The capsule is left behind after cataract removal and is the receptacle for the artificial lens implant. PCO occurs when lens epithelial cells in the so-called “equatorial” region of the lens divide and migrate inappropriately along the posterior capsule; this induces wrinkling and progressive opacification of the posterior capsule with marked loss of visual acuity.

There is therefore a major clinical need to remove lens epithelial cells from the inside of the capsule, prior to artificial lens implantation, in order to prevent subsequent regrowth and migration of these cells. Laser therapy and physical “scraping” are used in an attempt to remove lens epithelial cells but these methods do not reliably remove 100% of lens epithelial cells and are also associated with complications such as retinal detachment. Another approach is to remove a segment of the posterior capsule prior to lens implantation: however, this has been associated with leakage of lens epithelial cells into the posterior chamber of the eye which could have detrimental consequences.

The use of medicaments—applied locally at the time of the operation—would in theory be another way to kill lens epithelial cells. However, such compounds would be required to be selectively toxic to the lens epithelial cells while sparing other ocular cell types and tissues that would be exposed due to local diffusion of the drug. It would also be highly advantageous for these compounds to kill lens cells by a specialized biological process termed apoptosis. Apoptosis causes cells to die in a way that avoids escape of noxious intracellular contents such as enzymes that could cause damage to the intraocular tissue. Unfortunately, however, the vast majority of agents that cause apoptosis are not selective for particular cell types and would be anticipated to cause the death of other cells, in addition to lens epithelial cells, within the eye. Additionally it has been shown that lens cells resident on the capsule are resistant to common apoptogens.

WO96/06863 disclosed that opioid receptor ligands induce apoptosis selectively in tumour cells and also in lens epithelial cells due to common properties of “self-reliance”. In WO 96/06863 it was claimed that selective killing of lens epithelial cells by opioid receptor ligands would be medically useful in the peri-operative management of cataracts to prevent or treat lens epithelial cell re-growth over lens implants, that is, the complication of posterior capsule opacification (PCO). Coating of the lens implant with opioids as a method of preventing or treating PCO was claimed as a particular embodiment.

WO00/00599 disclosed that sigma receptor ligands, which are distinct from oploid receptor ligands, kill selected cell populations including tumour cells and inflammatory cells and are therefore of use in the treatment of cancer and inflammatory disease.

WO01/74359 disclosed that sigma ligands modulate the survival of microvascular endothelial cells and that sigma-1 antagonists have an anti-angiogenic effect whereas sigma-1 agonists have a pro-angiogenic effect, both classes of agent therefore being of use in particular medical contexts.

WO02/079779 describes a screening method to identify compounds that would selectively kill tumour, microvascular endothelial and inflammatory cells, whilst sparing many normal cell types. This invention disclosed a cell-selective rise in calcium in lens epithelial cells. The death of lens epithelial cells (cultured in isolation from lens capsule) in response to sigma antagonists such as rimcazole has also been disclosed (Christopher Gribbon's PhD Thesis, University of Dundee 2002; and Spruce et al. Cancer Research 2004 Vol. 64 4875-4886).

Nevertheless, the art has become more complex since these disclosures. It is now apparent that agents that kill primary cultures of lens epithelial cells by the process of apoptosis are not necessarily of use in vivo (in the clinical situation). This is because it has been discovered that the ocular lens capsule (the basement membrane on which lens epithelial cells reside) produces resistance to apoptosis induction by many apoptosis inducers (see for example Christopher Gribbon PhD Thesis University of Dundee 2002). This reveals that agents that are potent inducers of apoptosis when lens epithelial cells are cultured in isolation, fail to induce apoptosis in the same cells when these cells are in proximity to the anterior surface of an ocular lens capsule, in this case a bovine capsule. Importantly, it was discovered that apoptosis-inducing agents that are rescued by the capsule include sigma antagonists such as rimcazole. Rimcazole is substantially less able to induce apoptosis in lens epithelial cells in the presence of the bovine capsule. These findings therefore cast considerable doubt on the use of sigma receptor ligands as agents to induce apoptosis in lens epithelial cells and thereby prevent posterior capsule opacification. Thus, it remains a problem to find agents that will kill lens epithelial cells when in proximity to a lens capsule, whilst substantially not affecting/harming other cells types which are in close proximity.

It is amongst the objects of the present invention to obviate and/or mitigate at least one of the aforementioned disadvantages.

Without wishing to be bound by theory, the invention is based in part on reasoning by the present inventors that, since apoptosis inducers can be selectively rescued by different extracellular matrices (and basement membranes), a sigma-1 antagonist, such as rimcazole would not necessarily be antagonised by the human ocular lens capsule. Furthermore, the inventors also postulated that primary cultures of lens epithelial cells that have been dissociated from and then reintroduced to the lens capsule might behave differently from lens epithelial cells that have remained in proximity with the lens capsule throughout.

Thus, in a first aspect, there is provided use of a human capsular bag system for testing agents which selectively kill and/or inhibit human lens epithelial growth, wherein the human capsular bag system comprises a lens capsule within which lens epithelial cells remain, but from which the fibre cells of the lens have substantially been removed.

Removal of the capsular bag contents may be carried out, for example, by hydrodissection and phacoemulsification. Importantly, the above system comprises both the anterior and posterior faces of the lens capsule.

The inventors proved to be correct in their prediction that agents, such as sigma antagonists would behave differently towards lens epithelial cells when present within the human capsular bag system, rather than when cultured in proximity to an anterior lens capsule from a non-human species. Rimcazole, for example, potently induces cell killing in human lens epithelial cells when contained in the human capsular bag system. Importantly, it was also discovered that a subset of lens epithelial cells are particularly susceptible: cells derived from the equatorial region which divide and migrate and are therefore particularly responsible for lens epithelial regrowth.

The capsular bag model system is particularly useful in testing sigma ligands, such as sigma antagonists or agonists for their ability to selectively inhibit lens epithelial growth or to induce lens cell killing

Preferably, the test agent/sigma ligand is capable of inhibiting growth of the lens epithelial cells present in the lens capsule and more preferably capable of selectively killing the lens epithelial cells.

There is also provided a method of identifying an agent, such as a sigma ligand, e.g. a sigma-1 antagonist, potentially suitable for treating PCO, comprising the steps of:

a) providing a so-called capsular bag system comprising a human lens capsule from which the lens contents—principally lens fibre cells—have substantially been removed but which still contains adherent lens epithelial cells;

b) contacting a test agent, typically a sigma ligand, with said lens epithelial cells residing on said lens capsule; and optionally, lens epithelial cells cultured in the absence of a capsule; and

c) observing a reduction in viability or growth when said test agent, typically a sigma ligand is contacted with said lens epithelial cells in proximity to the lens capsule.

Candidate sigma ligands may first be identified by classical radioligand binding assays, such as disclosed in, for example, WO00/00599.

Typically, the test agents, such as sigma ligands may also be contacted with other cells such as, corneal endothelial and trabecular meshwork or other normal human cells such as fibroblasts grown at low passage in order to ensure the selectively of the test agent/sigma ligand, that is, that the test agent/sigma ligand does not substantially kill and/or prevent growth of cells other than lens epithelial cells. Normal cells—with which lens epithelial cells are to be compared—are defined for the purposes of the invention as cells that have normal (typical) properties of survival and growth regulation and that therefore exclude lens epithelial cells and microvascular endothelial cells. Tumour cells and inflammatory cells are also excluded as control cells. A cell-selective effect of an agent would be determined when normal cells show an increase in viable cell number compared to baseline (pre-treatment) cell numbers over a period of time, preferably over 48-72 hours when exposed to the test agent. In contrast, lens epithelial cells would show substantially no change or a reduction in viable cell numbers when exposed to the test agent administered at the same concentration and with the cells under similar culture conditions, in particular at a similar cell density. Assays to reveal cell-selective growth inhibition or killing (cytotoxicity) are described, for example, in Spruce et al. Cancer Research 2004 and are understandable to those skilled in the art.

Typically the human lens capsule of the present invention may be supported on a solid substrate such as a plastics or glass surface, e.g. the surface of a petri dish. Sigma ligands may be first identified as such by conducting, for example, a radioligand binding assay, such as described in WO 00/00599 to ascertain that the test agent is a sigma ligand, before carrying out the method as described above. Importantly, sigma radioligand binding assays determine only that an agent binds to sigma sites and not whether it acts as an agonist or antagonist in functional terms (whether it acts to stimulate or inhibit the receptor). In WO 00/00599 it was disclosed that different sigma receptor subtypes—or even binding pockets on the same receptor molecule—can act in opposite ways to either promote or suppress apoptosis (cell death). Given that a number of sigma ligands bind to both sigma-1 and sigma-2 sites (that act in opposition to regulate cell death), the balance of their functional activities at the two sites then becomes important. Without wishing to be bound by theory, the inventors propose that, when it comes to the identification of novel sigma ligands of therapeutic usefulness for PCO, the functional endpoint is what matters (selective inhibition of growth or induction of death in lens epithelial cells). Thus, all-encompassing sigma radioligand binding assays accompanied by functional growth inhibition/cell death assays in lens epithelial cells may lead to the identification of a therapeutically meaningful subset of sigma ligands.

Contacting may simply be carried out by pipetting a solution comprising the test agent//sigma ligand onto the surface of the lens capsule and/or into the lens capsule itself, or washing or otherwise bathing the lens capsule in a solution comprising the test agent/sigma ligand.

Observing a reduction in growth or induction of cell killing by the said test agent on lens epithelial cells may be carried out for example using a microscope, such as a phase contrast microscope. This can be carried out using an automated system. Typically, the lens epithelial cells would be viewed before and after contacting with the test agent, in order to ascertain the effectiveness of the test agent at inhibiting growth the lens epithelial cells. Desirably the test agent not only inhibits lens epithelial cell growth, but also kills the lens epithelial cells.

Conveniently the test agent may also be tested in a cell viability assay, such as described in Spruce et al Cancer Research 2004 in order to ascertain whether or not the test agent also inhibits the growth and/or kills other cell types. Desirably the test agent should not substantially inhibit the growth and/or kill other “normal” cell types, such as corneal endothelial, trabecular meshwork cells or fibroblasts.

Optionally, the test agents may also be tested in a lens cell culture viability assay as disclosed, for example, in Spruce et al Cancer Research 2004) in which lens epithelial cells are grown in culture i.e. not within the lens capsule.

The present invention therefore provides a method for detecting test agents, such as sigma ligands, which can selectively inhibit the growth and/or kill lens epithelial cells whilst substantially not inhibiting the growth and/or killing other normal cells, such as corneal endothelial cells, trabecular meshwork cells or fibroblasts.

It has been taught previously that so-called sigma-1 antagonists have a particular value as agents to induce tumour cell death (Spruce et al Cancer Research 2004). Sigma-1 antagonists are known as such in the art from a traditional pharmacological classification (based for example on inhibition of psychotic behaviour in animal models). Broadly speaking, the same class of agent (defined by traditional pharmacology) also causes selective antagonism of sigma-1 receptor-mediated repression of the death programme in tumour and microvascular endothelial cells. It is proposed herein that the same traditional pharmacological classification of sigma-1 antagonists defines at least one subset of sigma ligand that has therapeutic application in PCO.

Whereas sigma-1 antagonists will broadly have activity against tumor and lens epithelial cells, it is clear that there are differences in potency amongst this class of agent that do not always correlate with affinity of interaction with the sigma receptor in standard radioligand binding assays. For example, the agent BD-1047 is a highly selective and potent sigma-1 antagonist but is less potent than other sigma-1 antagonists in its tumour cell killing properties. It has therefore been proposed that the subcellular localisation of the sigma receptor may contribute to the degree of susceptibility to sigma ligands (Spruce et al Cancer Research 2004). Without wishing to be bound by theory, the present inventors postulated that differential subcellular localisation of the sigma receptor, or other factors, could lead to sigma ligands being differentially potent in lens epithelial cells compared to tumour cells. As disclosed in more detail hereinafter, this prediction has turned out to be correct since the aforementioned compound BD-1047 (a highly selective sigma-1 antagonist) is substantially more effective at killing lens epithelial cells compared to tumour cells. Specifically, the IC50 (the concentration of drug required to produce 50% growth inhibition) of BD-1047 for tumour cells is in the region 50-100 micromolar; in contrast, the IC50 for human lens epithelial cells (in the capsular bag system) is in the region of 10 micromolar. Thus, BD-1047 and/or other sigma-1 antagonists may be agents to treat PCO even though they may be much less effective anti-tumour agents.

Experiments with rimcazole have revealed an additional mechanism by which the sigma-1 antagonists act on lens cells. Concentrations of rimcazole that were sub- or semi-lethal caused an accumulation of melanin pigment granules in the lens cells. The melanin accumulation may be part of a differentiation programme that would also lead to an arrest of lens cells proliferation. Melanin accumulation could therefore act as a biomarker of response during or after a period of sigma antagonist treatment. The sigma-1 antagonist BD1047 also induced pigmentation in the lens cells, indicating that the response is specific to sigma ligands.

Thus, in a further aspect, there is provided a method of testing and/or ensuring the efficacy of a sigma ligand, typically a sigma-1 antagonist, in treating/preventing PCO, comprising the steps of:

a) providing a human capsular bag system, preferably of human origin, comprising a lens capsule to which lens epithelial cells are adherent and from which the original lens contents (fibre cells) have substantially been removed;

b) contacting a sigma ligand, typically a sigma-1 antagonist with said lens epithelial cells within said lens capsule and optionally cultured lens epithelial cells without the capsule present; and

c) detecting any increase in pigmentation in the lens epithelial cells within said lens capsule and/or in cultured lens epithelial cells.

It is to be appreciated that the above method may be carried out visually, by comparing a degree of pigmentation of the lens epithelial cells, before and after contacting with the sigma ligand. This may be a manual operation, or could, for example, be automated, using, for example, a CCD camera and appropriate software to compare changes in pigmentation between images. As mentioned above, any increase in pigmentation is postulated to be due to melanin production in the epithelial cells as a result of adding a sigma ligand, such as sigma-1 antagonist.

The above method may be carried out in vitro when screening for appropriate sigma ligands and/or in situ, when a patient has been operated on, to remove a cataract and to check/monitor that the sigma ligand (e.g. sigma-1 antagonist) is functioning appropriately to inhibit growth and/or kill lens epithelial cells.

The present invention also provides use of at least one sigma ligand, such as a sigma-1 antagonist for the manufacture of a medicament for preventing and/or treating posterior capsule opacification (PCO).

There is also provided a method of preventing and/or treating PCO comprising the steps of contacting lens epithelial cells within the lens capsule, said cells belonging to a patient who has undergone or who is undergoing cataract surgery, with at least one sigma ligand, such as a sigma-1 antagonist.

Preferred sigma ligands are Rimcazole and BD1047.

It is understood therefore that the sigma ligand may be administered before, during and/or after cataract surgery. Generally speaking the sigma ligand may be administered as a topical formulation. However, before and/or during surgery, the sigma ligand could, for example, be injected, or otherwise administered within the lens capsule. Oral administration, as for example with rimcazole, is also a possibility.

Throughout the specification mention is made to sigma ligands and sigma-1 antagonists in general. Many compounds are known and/or can be identified as being sigma ligands, by, for example, a sigma ligand binding assay such as described in WO 00/00599. Preferred sigma ligands include rimazole and related compounds as disclosed in U.S. Pat. No. 4,379,160 and AU 201630, BD1063, BD1047, AC915, IPAG, NE-100, haloperidol, reduced haloperidol, BD-1008, BMY 14802, although this is not to be construed as limiting. The present invention also encompasses the use of salts and solvates of appropriate sigma ligands and mixtures of sigma ligands, such as a sigma-1 antagonist and sigma-2 agonist, providing such mixtures result in the selective inhibition of growth and/or killing of lens epithelial cells when in situ.

The present invention will now be further described by way of example and with reference to the figures which show:

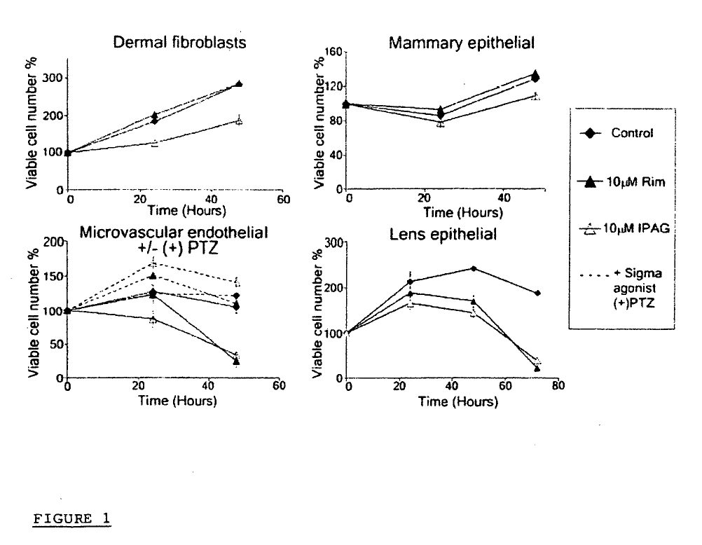

FIG. 1 shows than lens epithelial cells are sensitive to sigma 1 antagonists. Microvascular endothelial and lens epithelial cells resemble tumour cells in being susceptible to sigma-1 antagonists. Human adult male dermal fibroblasts, adult mammary epithelial cells, adult dermal microvascular endothelial cells and bovine lens epithelial cells at low passage were exposed to 10 μM concentrations of the sigma-1 antagonists rimcazole and IPAG for up to 72 hours. Change in cell viability over time was measured in the MTS assay; data points represent mean values (±SD), obtained from wells in triplicate, expressed relative to baseline (pre-treatment) values. Graphs depict representative experiments, performed at least three times. Microvascular endothelial cells were protected from rimcazole and IPAG by co-administration of equimolar concentrations of two prototypic sigma-1 agonists (+)-pentazocine (PTZ, dotted lines) and (+)-SKF10,047 (not shown).

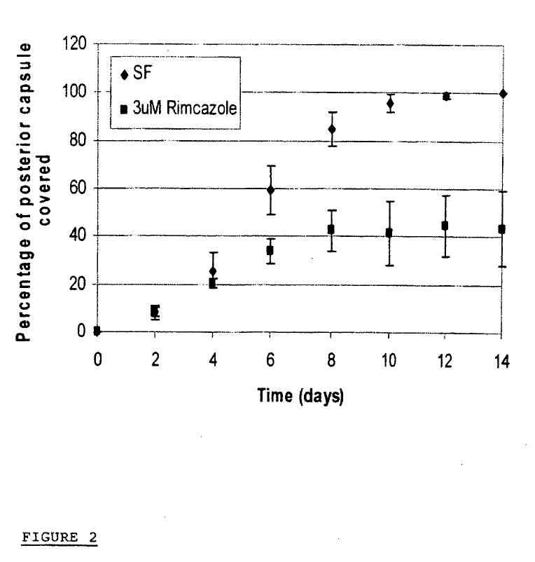

FIG. 2 shows that low concentrations of the sigma 1 antagonist Rimcazole inhibits growth of human lens epithelial cells on the posterior capsule. Rate of cell coverage of the posterior capsule beyond the rhexis; 100% represents confluency. Experiments were repeated on 4 occasions. The means and standard errors are display ed.

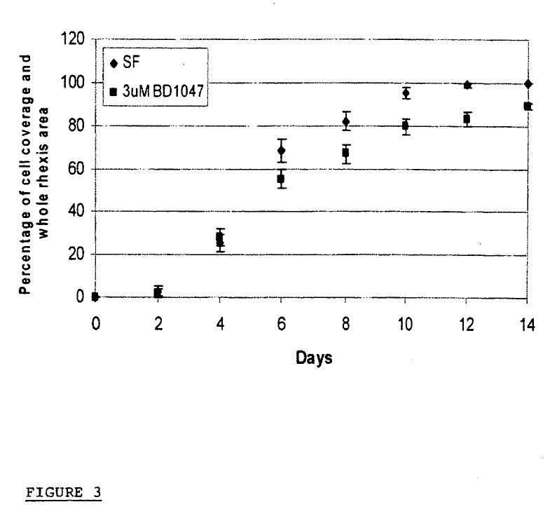

FIG. 3 shows that low concentrations (equal to rimcazole concentration used in FIG. 2) of the sigma 1 antagonist BI) 1047 inhibit growth of human lens epithelial cells on the posterior capsule. Rate of cell coverage of the posterior capsule beyond the rhexis; 100% represents confluency. Experiments were repeated on 4 occasions. The means and standard errors are displayed.

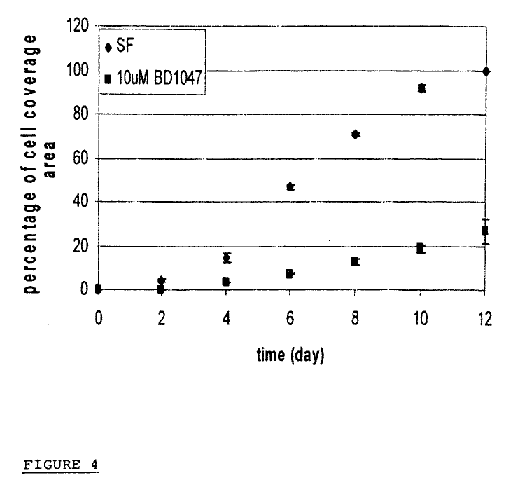

FIG. 4 shows that lens epithelial cells have a profile of relative susceptibility to sigma antagonists that differs from tumour cells; 10 micromolar concentrations of the sigma 1 antagonist BD 1047 (substantially less than concentrations of BD-1047 required to inhibit human tumour cell growth) markedly inhibit growth of human lens epithelial cells on the posterior capsule. Rate of cell coverage of the posterior capsule beyond the rhexis; 100% represents confluency. Experiments were repeated on 4 occasions. The means and standard errors are displayed.

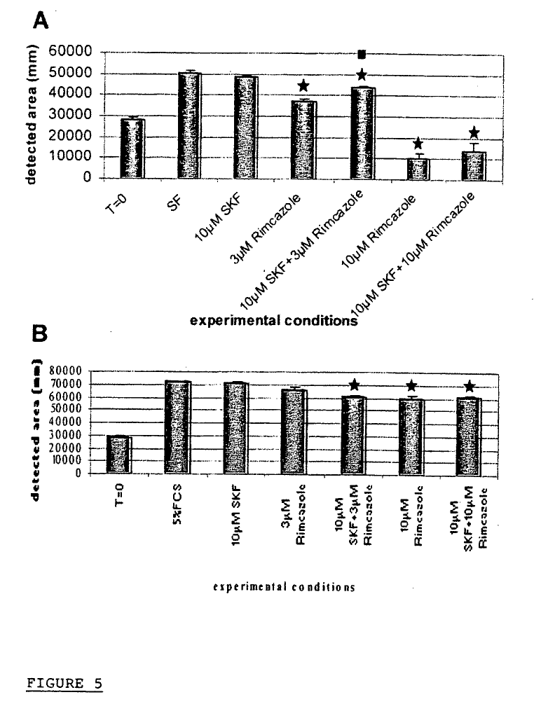

FIG. 5 shows that the growth of the spontaneously immortal lens cell line FHL124 is inhibited by the sigma 1 antagonist Rimcazole. The effect of sigma ligands on human lens cell growth in FHL124 cell line maintained in serum-free media (A) or EMEM supplemented with 5% FCS (B). The sigma agonist SKF10047 partially rescues the cells from Rimcazole inhibited growth at two concentrations. The cells were cultured for 4 days with experimental conditions. Experiments were repeated on 4 occasions. Data are expressed as Mean±S.E.M., the star represents significant difference from untreated control with rimcazole, and box represents significant difference from untreated control with (±)SKF 10047 (test p<0.05). Note that 124 cells do not behave same as native.

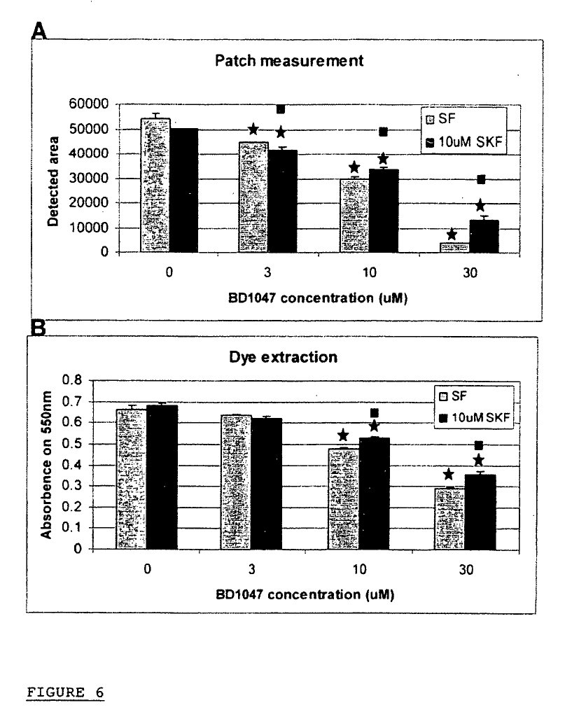

FIG. 6 shows that the growth of the spontaneously immortal lens cell line FHL124 is inhibited by the sigma 1 antagonist Rimcazole. Again SKF10047 partially inhibits the effect of Rimcazole. The effect of sigma-1 ligands on human lens cell growth in FHL 124 cell line maintained in serum-free media. A represents the data of patch area measurement. B represents the dye extraction from each stained patch, ie. cell number. The cells were cultured for 4 days with experimental conditions. Experiments were repeated on 4 occasions. Data are expressed as Mean±S.E.M, the star represents significant difference from untreated control with rimcazole, and ▪ represents significant difference from untreated control with (±)SKF10047 (test p<0.05).

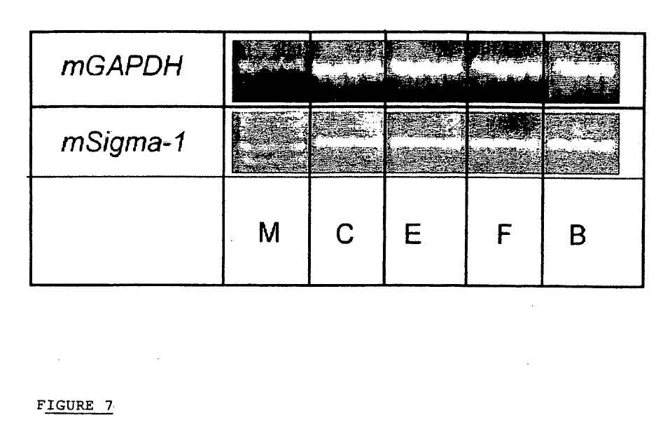

FIG. 7 shows that the sigma 1 receptor is expressed in the intact lens and epithelial cells migrating across the posterior capsule. RT-PCR agarose gels showing the expression of sigma-1 receptor mRNA in the central anterior epithelium (C), equatorial epithelium (E), fibre cells (F) and ex vivo capsular bag (B); M represents markers.

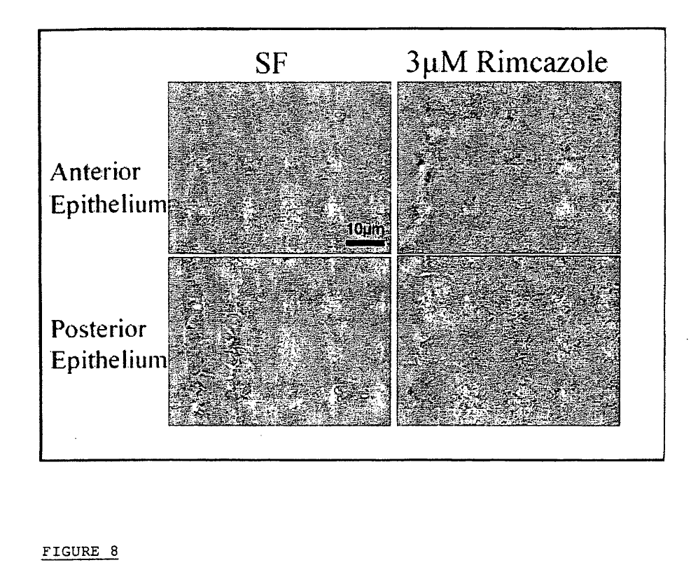

FIG. 8 shows that epithelial cells on the posterior capsule become pigmented in response to a sub-lethal dose of the sigma-1 antagonist Rimcazole. Phase micrographs of cultured human lens cells on central anterior (upper) and posterior capsule (lower) respectively. The cells were maintained in serum-free or supplemented (3 μM Rimcazole) media for one week. The images were converted to grayscale with Adobe photoshop software.

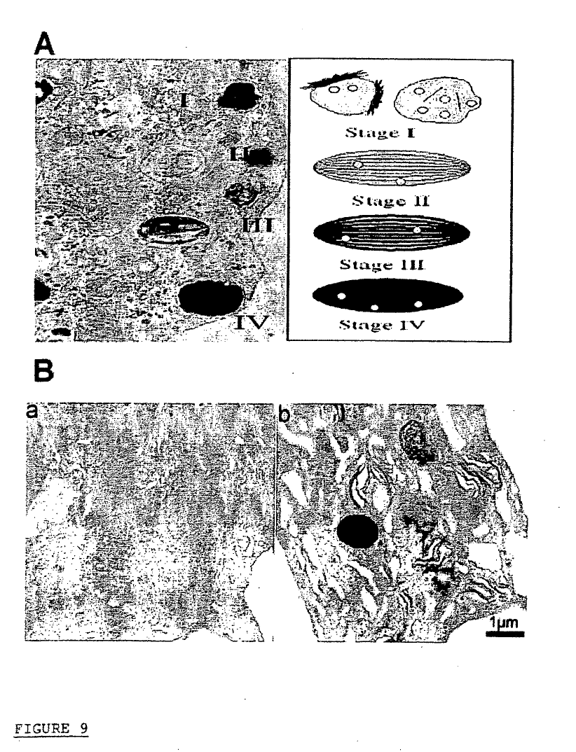

FIG. 9 shows that TEM confirms that the Rimcazole treated cells contain pigment granules at various stages of maturation. Formation of pigment granules in human epithelial cells. A. Different stages of pigment granule formation (I-IV) as defined in MNT-1 melanoma cells by Seiji et al. 1963. B. Cross section of human lens epithelial cells grown in (a) control medium and (b) the presence of 3 μM BD1047.

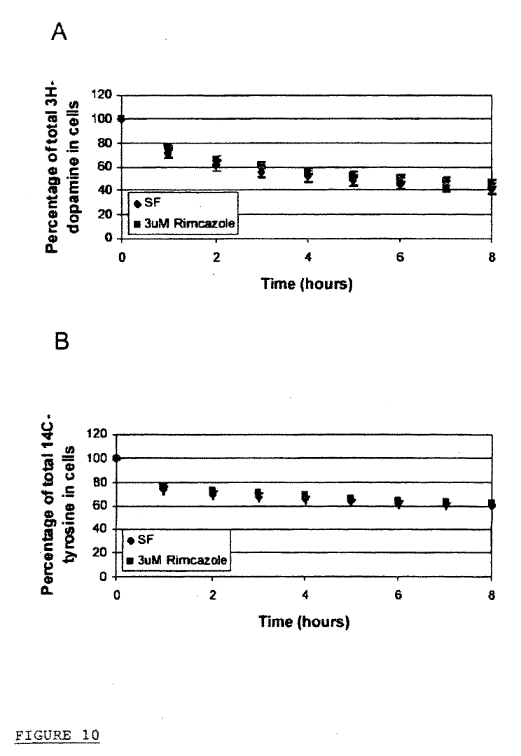

FIG. 10 shows that Rimcazole has no effect on the efflux of Dopamine or Tyrosine from lens cells. Efflux of (A) ³H-dopamine and (B) ¹⁴C-tyrosine from human lens epithelial cells in control (SF) and rimcazole supplemented media. Data are presented as mean±6 separate experiments.

### MATERIALS AND METHODS

### Anterior Lens Epithelium

The use of human tissue in the study was in accordance with the provisions of the Declaration of Helsinki. Human eye tissue donated for research was obtained from the East Anglian Eye Bank and the lens dissected from zonules and placed anterior side down onto a sterile 35-mm tissue culture dish. The area of the central anterior epithelium and underlying capsule was then carefully dissected out and transferred to a fresh 35 mm culture dish where it was secured with pins.

In vitro Capsular Bag Model

The model previously described by Liu et al (1996) was used. A sham cataract operation was performed on human donor eyes. The resultant capsular bag was then dissected free of the zonules and secured on a sterile 35 mm PMMA petri dish. Eight entomological pins (D1: Watkins and Doncaster Ltd., Kent, UK) were inserted through the edge of the capsule to retain its circular shape. Incubation was at 35° C. in a 5% CO₂ atmosphere. Ongoing observations were performed with a Nikon phase-contrast microscope and images captured with a digital camera (Coolpix 950; Nikon. Tokyo, Japan) with associated imaging software (Mr Y Zhu, personal communication). In some cases preparations were used for radioactive isotope studies.

### Growth Assay

Capsular bags were dissected and donor pairs checked for comparable cell coverage of the remaining anterior capsule by phase-contrast microscopy (see Liu et al for details). The bags were maintained in Eagle's minimum essential medium (EMEM) or EMEM supplemented with either 3 μM Rimcazole dihydrochloride (rimcazole), 10 μm(+)-SKF 10047 hydrochloride (SKF), and 3 μM or 10 μM BD1047 dihydrochloride (BD1047) (all supplied by TOCRIS) and incubate at 35° C. in a 5% CO₂ atmosphere. The medium was replaced every 2 days and ongoing observations and analyses were performed as above.

### Western Blot Analysis

After dissection, epithelial preparations were washed in serum-free (SF) EMEM then maintained in fresh EMEM and EMEM containing 3 μM Rimcazole dihydrochloride (rimcazole) for 5 days. Cells were then lysed on ice in buffer: 50 mM HEPES [pH 7.5], 150 mM NaCl, 1% Triton X-100, 1 mM EDTA, 10% glycerol, 10 mM, sodium pyrophosphate, 2 mM sodium orthovanadate, 10 mM sodium fluoride, 1 mM phenylmethylsulfonyl fluoride (PMSF), and 10 μg/ml aprotinin. Lysates were pre-cleared by centrifuging at 1300 rpm 4° C. for 10 minutes, and the protein content of the soluble fraction was assayed by A Bicinchoninic acid protein assay (Pierce, Rockford, Ill.). Equal amounts of protein from each sample were loaded onto 10% SDA-PAGE gels for electrophoresis and transfer onto polyvinylidene difluoride (PVDF) membrane (NEN Life Science Products, Boston. MA) with a semidry transfer cell (Trans-Blot; Bio-Rad, Herts. UK). Proteins were detected using a chemiluminescent blot analysis system (ECL⁺ Amersham Biosciences, Amersham, UK) with Anti-tyrosinase (Upstate Biotechnology, Lake Placid, N.Y.), Anti-tyrosinase related protein 1 (TRP1) (SANTA CRUZ BIOTECHNOLOGY, INC), Anti-tyrosinase related protein 2 (TRP2) (SANTA CRUZ BIOTECHNOLOGY. INC) and Anti-Actin (SANTA CRUZ BIOTECHNOLOGY, INC).

### Reverse Transcription-Polymerase Chain Reaction

After dissection, epithelial preparations were washed in serum free EMEM, and RNA was collected from the cells by using a mini kit (RNeasy; Qiagen Ltd., Crawley, UK), RNA (250 ng) was reverse transcribed in a 20 μl reaction mixture (Superscript™ II RT: Invitrogen Ltd. Paisley, UK). cDNA (1 μl; diluted 1 in 5 in sterile double distilled water) was amplified by PCT in a 20 μl reaction buffer in the following condition: 0.5 μM each primer (Invitrogen Ltd, Paisley, UK), 0.8 mM deoxy-nucleoside trisphosphate mixture (Biolin Ltd. London, UK), 10 mM Tris-HCl, 1.5 mM MgCl₂, 50 mM KCl and 2.5 U TaqDNA polymerase (Roche Diagnostics, Lewes, UK). PCR was performed using the following program with a thermal controller (MJ Research Inc. Reno. NV): initial denaturation 95° C. for 2 minutes; denaturation at 94° C. for seconds; annealing at 55° C. for 30 seconds; extension at 72° C. for 40 seconds. Step 2 through 4 were cycled 27 times (GAPDH); 36 cycles (sigma 1 receptor) with a final extension at 72° C. for 10 minutes. The oligonucleotide primer (5′-3′) sequences specific for the genes examined were as follows:

GAPDH: ACCACAGTCCATGCCATCAC (sense) and TCCACCACCCTGTTGCTGTA (anti-sense); Sigma-1 receptor: 5′-AGCGCGAAGAGATAGC-3′ (sense) and 5′-AGCATAGGAGCGAAGAGT-3′ (anti-sense). PCT products, together with the 100 bp DNA markers (Invitrogen-life Technologies), were run on a 1% agarose gel, and images were captured and analysed (1D system; Eastman Kodak, Rochester, N.Y.).

### Measurement of Radioactive Isotopes

Capsular bags were maintained in Eagle's minimum essential medium (EMEM) or EMEM supplemented with 3 μM. Rimcazole dihydrochloride (rimcazole) (TOCRIS) for 7 days (when the pigment granules could be observed in cells). 1 μCl/ml of ³H-dopamine and 2 μCi/ml of ¹⁴C-tyrosine (Amersham Biosciences) were then added to each dish for an additional 24 hours. At the end point, 10 μl of medium were collected from each dish, and transferred into scintillation vials. The bags were washed briefly twice with EMEM and fresh control or experimental media added for one hour. The medium was then collected into scintillation vials and the procedure repeated eights times. The experiment was then terminated by adding 1 ml of ice-cold 5% Trichloroacetic acid (TCA) (Fisher Chemicals) to each dish. After 30 minutes, the TCA was removed from each well to determine the cytosolic dopamine and tyrosine levels. 1 ml of 250 mM NaOH was then added to each dish to determine the radio-isotope levels in the TCA precipitable fraction. 10 mls of scintillation fluid (Hisafe Supermix) were then added to each scintillation vial and the samples assayed using a Wallac scintillation counter with appropriate background controls. Results were expressed in disintegrations per minute (DPM).

### Experimental Results

### In Vitro Cell Death Assays

The present inventors have tested a panel of sigma-1 receptor antagonists such as IPAG, BD1047, BD-1063 and BMY 14802 in addition to Rimcazole for their ability to kill lens cells grown on tissue culture plastic plates. These have been compared to the effect of these agents on other cell types, such as tumour cells, previously shown to be susceptible to sigma-1 antagonists as well as primary cells which are resistant to these drugs. Primary bovine lens cells in tissue culture are almost as susceptible to Rimcazole and IPAG as tumour cells (FIG. 1).

### Capsular Bag Assays

Rimcazole inhibits growth of human lens cells on the posterior capsule in the present PCO model at doses comparable or less than the dose required to kill tumour cells (3 μm: FIG. 2). BD1047 at this dose is less inhibitory but at 10 μm (still less than is needed to kill tumour cells) it potently inhibits growth of lens cells on the capsule (FIG. 4).

### Cell Growth Assay

The growth of the spontaneously immortal human lens cell line FHL124 was investigated using a patch assay. Cells grown from a single patch derived from a coverslip and stained with dye to estimate the cell number. At both 3 and 10 μm Rimcazole growth is significantly inhibited in serum free medium but in 5% FCS growth inhibition is less marked at the higher dose (FIG. 5). The specific sigma-1 agonist (+)—SKF1004 partially rescues sigma antagonist-mediated growth inhibition at both concentrations in serum free medium (FIG. 5). (+)-SKF10047 also partially restores growth to BD1047 inhibited FHL124 cells at the higher concentrations (10 and 30 μm). (FIG. 6). BD1047 is less effective at the lower dose (10 μm) most of its effects seem to be on patch size rather than protein content (i.e. cell number, FIG. 6). Attenuation of rimcazole and BD-1047-mediated growth inhibition by a highly specific sigma-1 agonist confirms that growth inhibition is at least partly mediated through antagonism of sigma-1 sites.

### Expression of Sigma-1 Receptors in the Lens

RT-PCR demonstrates sigma-1 receptor m-RNA in all regions of the human lens: both in the anterior and equatorial epithelium and in the fibre cells. Additionally epithelial cells migrating across the posterior capsule in the capsular bag model also express sigma-1 receptors (FIG. 7).

Effects of Sigma antagonists on pigmentation of lens cells Observation of primary human lens epithelial cells on the posterior capsule during growth inhibition assays with the sigma antagonists Rimcazole and BD1047 showed that these cells become pigmented while control cells remained clear. This pigmentation was also present in cells similarly tested on the rhexis (anterior capsule) removed during preparation of the capsular bag (FIG. 8) and in the FHL-124 lens cell line (not shown). Transmission electron microscopy of these cells showed that the pigment was packaged into vesicles (FIG. 10B) and appeared to follow the same developmental progression (stages I-IV) as in MNT-1 melanoma cells (FIG. 9A, Rapaso et al, 2002; Seiji et al. 1963) suggesting that the pigment was melanin. Sigma receptor antagonists have been reported to inhibit dopamine transport (Moison et al. 2003: Izenwasser. S. et al, 1993; Nuwayhid and Werling, 2003) but dopamine efflux was unaffected by Rimcazole in the capsular bag system (FIG. 10A).

### Discussion

The results demonstrate that lens epithelial cells are very sensitive to sigma antagonists and that this sensitivity is retained in cultured cells derived from the epithelium. Sigma antagonists can induce cell death and inhibit cell growth both in culture and on the posterior capsule. Sigma antagonists can thus limit cell coverage of the posterior capsule in the capsular bag model of PCO. The growth inhibitory effect of both rimcazole and the specific sigma-1 antagonist BD-1047 is opposed by the sigma-1 agonist (+)-SKF10,047. This confirms that the growth inhibitory effect of rimcazole and BD-1047 is mediated at least in part by antagonism at sigma-1 sites. This is also supported by expression of the sigma-1 receptor in all regions of the intact lens including the fibre cells and its expression is retained by the epithelial cells on the posterior capsule (a position which they occupy only after cataract extraction).

Both the sigma-1 specific antagonist BD-1047 and Rimcazole induce pigmentation in the lens cells if administered at a sub-lethal dose. This pigmentation comprises melanin granules of the type seen in melanocytes and is likely to be the result of the upregulation of two enzymes in the melanin synthetic pathway, Tyrosinase and TYRP1, while TRP2 is unaffected, (data not shown). Thus lens epithelial cells have the ability to make pigment which may be suppressed by agonistic signalling at the sigma-1 receptor.

The sigma antagonists used in this study are potential candidates for the treatment of PCO and as lead compounds for the development of effective strategies for this common problem.

Melanin accumulation may be used as a marker of transdifferentiation of lens epithelial cells; thus, melanin induction could be a marker of cell cycle exit—that is to say, melanin induction is consistent with proliferation inhibition which, along with apoptosis induction would be a desirable outcome in the context of therapy for PCO.

Together these data suggest therefore that a subset of agents that may be particularly useful for treatment of PCO would be sigma ligands that, when administered at sublethal concentrations induce melanin (a marker of proliferation inhibition) within lens epithelial cells. Such agents may be identified by a combination of standard radioligand binding assays to identify sigma receptor binding activity (as described in WO 00/00599) and the induction of melanin within cultured lens epithelial cells (using the techniques described herein).

Advantageously the determination of melanin accumulation in residual lens epithelial cells may be used as biomarker of response to rimcazole and/or other test agents of the invention. Such an assessment would be useful as an efficacy surrogate for clinical trials in PCO.

### REFERENCES

Gribbon, C PhD Thesis University of Dundee 2002.
Liu C S, Wormstone I M, Duncan G. Marcantonio J M, Webb S F. Davies P D. A study of human lens cell growth in vitro. A model for posterior capsule opacification. Invest Ophthalmol Vis Sci. 1996 April; 37(5):906-14.
M. Seiji, S. Iwashita, J Biochem (Tokyo) 54, 465 (November, 1963).
Rapaso, G. et al. 2002 Cell Struct. Funct. Vol. 27 pp 443-456.
Moison et al. 2003; Neuropharmacol. Vo. 45 pp 945-953.
Izenwasser, S. et al 1993 Eur. J. Pharmacol. Vol. 243 pp 201-205.
Nuwayhid, S. J. and Werling, L. L. 2003 J. Pharmacol. Exp. Ther. Vol. 306 pp 934-940.
Spruce et al 2004 Cancer Research Vol. 64 4875-4886

1-17. (canceled)

18. A method of identifying an agent, potentially suitable for treating PCO, comprising the steps of:
a) providing a capsular bag system comprising a human lens capsule from which the lens contents—principally lens fibre cells—have substantially been removed but which still contains adherent lens epithelial cells;
b) contacting a test agent, with said lens epithelial cells residing on said lens capsule; and optionally, lens epithelial cells cultured in the absence of a capsule; and
c) observing a reduction in viability or growth when said test agent, is contacted with said lens epithelial cells in proximity to the lens capsule.

19. The method according to claim 18 wherein the test agent is a sigma ligand.

20. The method according to claim 18 wherein the test agent is a sigma-1 antagonist.

21. The method according to claim 18 wherein the test agent is also conducted with other cells, such as corneal endothelial and trabecular mesh work or other normal human cells in order to ensure the selectivity of the test agent.

22. The method according to claim 18 wherein the lens capsule is supported on a solid substrate.

23. The method according to claim 18 wherein contacting is carried out by pipetting a solution comprising the test agent onto the surface of the lens capsule and/or into the lens capsule itself, or washing or otherwise bathing the lens capsule in a solution comprising the test agent.

24. The method according to claim 18 wherein observing a reduction in growth or induction of cell killing by the said test agent on the lens epithelial cells, is carried out using a microscope.

25. The method according to claim 18 wherein the test agent is also tested in a lens cell culture viability assay.

26. A method of testing and/or ensuring the efficacy of a sigma ligand, in treating/preventing PCO, comprising the steps of:
a) providing a human capsular bag system, comprising a lens capsule to which lens epithelial cells are adherent and from which the original lens contents (fibre cells) have substantially been removed;
b) contacting a sigma ligand, with said lens epithelial cells within said lens capsule and optionally cultured lens epithelial cells without the capsule present; and
c) detecting any increase in pigmentation in the lens epithelial cells within said lens capsule and/or in cultured lens epithelial cells.

27. The method according to claim 26 wherein the sigma ligand is a sigma-1 antagonist.

28. The method according to claim 26 wherein the detection of any increase in pigmentation in the lens epithelial cells is carried out by an automated system, using, for example, a CCD camera and appropriate software to compare changes in pigmentation between images.

29. The method according to claim 26 is carried out in vitro or in vivo.

30. A method of preventing and/or treating PCO comprising the steps of contacting lens epithelial cells within the lens capsule, said cells belonging to a patient who has undergone or who is undergoing cataract surgery, with at least one sigma ligand.

31. The method according to claim 30 wherein the sigma ligand is a sigma-1 antagonist.

32. The method according to claim 30 wherein the sigma ligand is rimcazole or BD1047.


SEQUENCE LISTING (4 sequences)

SEQ ID NO: 1 - DNA, 20 bp, Artificial
  RT-PCR primer
accacagtccatgccatcac

SEQ ID NO: 2 - DNA, 20 bp, Artificial
  RT-PCR primer
tccaccaccctgttgctgta

SEQ ID NO: 3 - DNA, 16 bp, Artificial
  RT-PCR primer
agcgcgaagagatagc

SEQ ID NO: 4 - DNA, 18 bp, Artificial
  RT-PCR primer
agcataggagcgaagagt